(19) United States
(12) Patent Application Publication
(10) Pub. No.: US 2025/0387102 A1
(43) Pub. Date: Dec. 25, 2025

(54) METHOD FOR PROVIDING INFORMATION ON SEVERITY OF AORTIC STENOSIS AND DEVICE USING THE SAME

(71) Applicant: ONTACT HEALTH CO., LTD., Seoul (KR)

(72) Inventors: Seung Ah Lee, Seoul (KR); Jae Ik Jeon, Gyeonggi-do (KR); Ji Yeon Kim, Seoul (KR)

(73) Assignee: ONTACT HEALTH CO., LTD., Seoul (KR)

(21) Appl. No.: 19/242,778

(22) Filed: Jun. 18, 2025

(30) Foreign Application Priority Data
Jun. 19, 2024 (KR) 10-2024-0079938
Feb. 21, 2025 (KR) 10-2025-0022726

(51) Int. Cl. A61B 8/00; A61B 8/08; G06T 7/10; G06T 7/62; G16H 50/20; G16H 50/30

(52) U.S. Cl. CPC A61B 8/5223; A61B 8/0883; G06T 7/10; G06T 7/62; G16H 50/20; G16H 50/30; G06T 2207/10132; G06T 2207/20081; G06T 2207/20112; G06T 2207/30048; G06T 2207/30168

(57) ABSTRACT

The present disclosure provides a method for providing information on severity of aortic stenosis implemented by a processor, the method including receiving a cardiac ultrasound image of a subject, extracting features from the received cardiac ultrasound image using a prediction model trained to predict the severity of the aortic stenosis by inputting the cardiac ultrasound image, and determining the severity of the aortic stenosis for the subject based on the extracted features using the prediction model, and provides a device using the same.

### CROSS-REFERENCE TO RELATED APPLICATIONS

This application claims priority to Korean Patent Application No. 10-2024-0079938, filed Jun. 19, 2024, and to Korean Patent Application No. 10-2025-0022726, filed Feb. 21, 2025, both of which are incorporated by reference in entirety for all purposes. The USPTO is invited to retrieve the priority documents using the provided DAS codes.

### TECHNICAL FIELD

The present disclosure relates to a method for providing information on the severity of aortic stenosis and a device for providing information on severity of aortic stenosis using the method.

### BACKGROUND

Aortic stenosis (AS) is a heart disease that occurs when an aortic valve area becomes narrow, which causes resistance to a blood flow from a left ventricle to an aorta, and the left ventricle wall may become thicker. When the stenosis is severe, symptoms such as shortness of breath during exercise, chest pain, fainting, fatigue, and growth disorders may occur.

Causes of the aortic stenosis include degenerative calcification of the valve due to aging, congenital abnormalities of the valve, rheumatic valve disease, hypertrophic cardiomyopathy, and other heart diseases, which can block the left ventricular outlet.

Meanwhile, cardiac ultrasound is a major diagnostic tool for evaluating the aortic stenosis and severity thereof, and can help to evaluate the structure and function of the heart and accurately determine the location and degree of stenosis. In particular, the cardiac ultrasound can measure an electrical activity of the heart and evaluate the degree of stenosis of the valves and the function of the left ventricle.

However, the evaluation for the diagnosis and severity of the aortic stenosis using the cardiac ultrasound is dependent on interpretation of the medical staff, and the reliability of the results may also depend on the skills of the medical staff.

Accordingly, there is a continuous need for the development of a new information providing system capable of deriving highly accurate information from a cardiac ultrasound image that can provide reliable information regarding the aortic stenosis, especially the severity thereof.

The background technology of the disclosure has been written to facilitate understanding of the present disclosure. It should not be understood that the matters described in the background technology of the disclosure are recognized as prior art.

### BRIEF DESCRIPTION OF DRAWINGS

The above and other aspects, features and other advantages of the present disclosure will be more clearly understood from the following detailed description taken in conjunction with the accompanying drawings, in which:

FIG. 1 illustrates a system for providing information on severity of aortic stenosis using a device for providing information on the severity of aortic stenosis according to one embodiment of the present disclosure;

FIG. 2A is a block diagram illustrating the configuration of a medical staff device according to one embodiment of the present disclosure;

FIG. 2B is a block diagram illustrating the configuration of a server for providing information according to one embodiment of the present disclosure;

FIGS. 3A to 3C illustrate a procedure of a method for providing information on the severity of aortic stenosis according to one embodiment of the present disclosure;

FIGS. 4A and 4B and FIG. 5 illustrate the procedure of the method for providing information on the severity of aortic stenosis according to one embodiment of the present disclosure;

FIG. 6 illustrates an example of a procedure of a method for providing information on the severity of aortic stenosis according to another embodiment of the present disclosure; and

FIGS. 7A and 7B illustrate evaluation results of a prediction model used in the method for providing information according to various embodiments of the present disclosure.

### DETAILED DESCRIPTION

To solve the above-mentioned problem, the inventors of the present disclosure have attempted to develop an information providing system based on an artificial neural network trained to predict the severity of aortic stenosis for cardiac ultrasound images.

In particular, the inventors of the present disclosure have attempted to build a prediction model based on an artificial neural network that can predict the severity of the aortic stenosis using only the cardiac ultrasound image without going through a process of comparing the degree of stenosis based on cardiac ultrasound measurements with guideline criteria in determining the severity.

As a result, the inventors of the present disclosure have developed a prediction model that can extract features from the cardiac ultrasound images and predict the severity of the aortic stenosis from the extracted features.

Meanwhile, the inventors of the present disclosure have noted that it is possible to obtain higher diagnostic performance when structural features of the anatomical structure of the heart in the cardiac ultrasound image are reflected to predict the severity of the aortic stenosis in the prediction model.

In this regard, the inventors of the present disclosure have paid more attention to the fact that the severity of the aortic stenosis progresses serially and have attempted to reflect measurement parameters that can reflect the features in predicting the severity of aortic stenosis.

More specifically, the inventors of the present disclosure have constructed a network module capable of predicting the severity of aortic stenosis using only B-mode images, without conversion to Doppler mode or M-mode cardiac ultrasound images.

Meanwhile, the inventors of the present disclosure have constructed a prediction model to output a severity score corresponding to the severity and have recognized that it is possible to provide information on a more detailed severity level for a subject by providing the severity score.

The inventors of the present disclosure have expected that by applying the newly constructed artificial neural network, the clinical process can be supplemented to enable prediction of the severity without a comparison procedure with known severity guidelines, and that highly reliable information may be provided.

Furthermore, the inventors of the present disclosure have recognized that by applying a new artificial neural network, it is possible to solve the problem of the conventional process in which the reliability of the diagnosis result of the severity of aortic stenosis may vary depending on the skill level.

Accordingly, the inventors of the present disclosure have expected that by providing a new information providing system, it is possible to provide highly reliable analysis results for cardiac ultrasound images regardless of the skill level of the medical staff.

Accordingly, an object of the present disclosure is to provide a method for providing information on severity of aortic stenosis, which extracts features from a received cardiac ultrasound image using an artificial neural network-based prediction model and provides information on the severity of aortic stenosis, and a device and system using the same.

Objects of the present disclosure are not limited to the objects mentioned above, and other objects not mentioned will be clearly understood by those skilled in the art from the description below.

To solve the above-described objects, a method for providing information on severity of aortic stenosis according to one embodiment of the present disclosure is provided. The method is a method for providing information on severity of aortic stenosis implemented by a processor, the method including: receiving a cardiac ultrasound image of a subject; extracting features from the received cardiac ultrasound image using a prediction model trained to predict the severity of the aortic stenosis by inputting the cardiac ultrasound image; and determining the severity of the aortic stenosis for the subject based on the extracted features using the prediction model.

In at least one implementation, the method may further include: after the extracting the features, segmenting an anatomical structure of a heart based on the extracted features using the prediction model; and determining the severity of the aortic stenosis for the subject based on the segmented anatomical structure using the prediction model.

In at least one implementation, the method may further include: after the extracting the features, segmenting an anatomical structure of the heart based on the extracted features using the prediction model; determining a cardiac measurement based on the extracted features using the prediction model; and determining the severity of the aortic stenosis for the subject based on the segmented anatomical structure and the cardiac measurement using the prediction model.

In at least one implementation, the cardiac measurement may be at least one of Vmax (peak aortic jet velocity), MPG (mean pressure gradient), AVA (aortic valve area), EOA (effective orifice area), DI (dimensionless index) and indexed AVA, and the prediction model may include at least one network module trained to determine at least one cardiac measurement by inputting the feature.

In at least one implementation, a plurality of the network modules may be provided, the prediction model may further include a fusion layer and an output layer, and the method may further include fusing cardiac measurements determined from each of the plurality of network modules through the fusion layer and outputting and determining the severity of aortic stenosis based on the fused cardiac measurement results through the output layer.

In at least one implementation, the cardiac ultrasound image may be a B-mode PLAX (parasternal long-axis) cross-sectional image or a B-mode PSAX (parasternal short-axis) AV (aortic valve) level cross-sectional image.

In at least one implementation, the method may further include: after receiving, evaluating quality of the cardiac ultrasound image; and providing a guideline to acquire the B-mode PLAX cross-sectional image or the B-mode PSAX AV level cross-sectional image, depending on the evaluated quality of the cardiac ultrasound image.

In at least one implementation, determining the severity of the aortic stenosis may include determining a severity score corresponding to the severity of the aortic stenosis using the prediction model.

To solve the problem as described above, a method for providing information on severity of aortic stenosis according to another embodiment of the present disclosure is provided. A method for providing information on severity of aortic stenosis implemented by a processor, includes: receiving a cardiac ultrasound image of a subject; extracting features from the received cardiac ultrasound image using a prediction model trained to predict the severity of the aortic stenosis by inputting the cardiac ultrasound image; segmenting an anatomical structure of a heart based on the extracted features using the prediction model; determining a cardiac measurement based on the extracted features; and determining the severity of the aortic stenosis for the subject based on the segmented anatomical structure and the cardiac measurement using the prediction model.

To solve the problem as described above, a device for providing information on severity of aortic stenosis according to still another embodiment of the present disclosure is provided. The device includes: a communication unit configured to receive a cardiac ultrasound image of a subject; and a processor functionally connected to the communication unit. In this case, the processor is configured to extract features from the received cardiac ultrasound image using a prediction model trained to predict the severity of aortic stenosis by inputting a cardiac ultrasound image to determine the severity of the aortic stenosis for the subject based on the extracted features using the prediction model.

In at least one implementation, the processor may be further configured to segment an anatomical structure of the heart based on the extracted features using the prediction model to determine the severity of the aortic stenosis for the subject based on the segmented anatomical structure using the prediction model.

In at least one implementation, the processor may be configured to segment an anatomical structure of the heart based on the extracted features using the prediction model, determine a cardiac measurement based on the extracted features using the prediction model, and determine the severity of the aortic stenosis for the subject based on the segmented anatomical structure and the cardiac measurement using the prediction model.

In at least one implementation, a plurality of the network modules may be provided, the prediction model may further include a fusion layer and an output layer, and the processor may be further configured to fuse the cardiac measurements determined from each of the plural network modules through the fusion layer and output and determine the severity of the aortic stenosis based on the fused cardiac measurement results through the output layer.

In at least one implementation, the processor may be further configured to evaluate quality of the cardiac ultrasound image and provide a guideline to acquire a B-mode PLAX cross-sectional view or a B-mode PSAX AV level cross-sectional view, depending on the evaluated quality of the cardiac ultrasound image.

In at least one implementation, the processor may be further configured to determine a severity score corresponding to the severity of the aortic stenosis using the prediction model.

In order to solve the problem as described above, a device for providing information on severity of aortic stenosis according to still another embodiment of the present disclosure is provided. The device includes: a communication unit configured to receive a cardiac ultrasound image of a subject; and a processor functionally connected to the communication unit. In this case, the processor is configured to extract features from the received cardiac ultrasound image using a prediction model trained to predict the severity of the aortic stenosis by inputting the cardiac ultrasound image, segment an anatomical structure of the heart based on the extracted features using the prediction model, determine a cardiac measurement based on the extracted features using the prediction model, and determine the severity of the aortic stenosis for the subject based on the segmented anatomical structures and the cardiac measurement using the prediction model.

To solve the aforementioned problem, a system for providing information on severity of aortic stenosis according to still another embodiment of the present disclosure is provided.

The system includes an internal memory configured to store a cardiac ultrasound image of a subject and a prediction model trained to predict the severity of the aortic stenosis by inputting the cardiac ultrasound image, and is configured to access the internal memory, extract features from a received cardiac ultrasound image using the prediction model, segment an anatomical structure of the heart based on the extracted features using the prediction model, determine a cardiac measurement based on the extracted features, and determine the severity of the aortic stenosis for the subject based on the segmented anatomical structure and the cardiac measurement using the prediction model.

Specific details of other embodiments are included in the detailed description and drawings.

At least one implementation provides an information providing system for the severity of aortic stenosis based on an artificial neural network capable of providing information on the severity of the aortic stenosis using the cardiac ultrasound images.

At least one implementation provides an information providing system based on a prediction model that can predict the severity of the aortic stenosis using the cardiac ultrasound image without going through a process of comparing the degree of stenosis based on the cardiac ultrasound measurements with guideline criteria, thereby providing faster and more accurate diagnostic results.

At least one implementation provides the prediction model configured to output the severity score, thereby enabling the provision of more detailed information on the severity level for a subject.

At least one implementation provides highly reliable analysis results for the cardiac ultrasound image regardless of a skill level of the medical staff and can contribute to establishing more accurate decision-making and treatment plans at the image analysis stage.

The effects of various embodiments and implementations are not limited to those exemplified above, and further diverse effects are included in the present specification.

The effects of various embodiments and implementations are not limited to the aforementioned effects, and other effects, which are not mentioned above, will be apparently understood to a person having ordinary skill in the art from the following description.

The objects to be achieved by various embodiments and implementations, the means for achieving the objects, and the effects of various embodiments and implementations here do not specify essential features of the claims, and, thus, the scope of the claims is not limited to the disclosure of the present disclosure.

Hereinafter, the exemplary embodiment of the present disclosure will be described with reference to the accompanying drawings and exemplary embodiments as follows. Scales of components illustrated in the accompanying drawings are different from the real scales for the purpose of description, so that the scales are not limited to those illustrated in the drawings.

The advantages and features of various embodiments and implementations, and the methods for achieving them, will become clear with reference to the embodiments described in detail below together with the attached drawings. However, the present disclosure is not limited to the embodiments disclosed below, but may be implemented in various different forms, and the present embodiments are provided only to make the disclosure of the present disclosure complete and to fully inform a person having ordinary skill in the art to which the present disclosure belongs of the scope of the disclosure

The shapes, sizes, ratios, angles, numbers, or the like disclosed in the drawings for explaining embodiments of the present disclosure are exemplary, and therefore, the present disclosure is not limited to the matters illustrated. In addition, in describing the present disclosure, if it is determined that a detailed description of a related known technology may unnecessarily obscure the gist of the present disclosure, the detailed description will be omitted. When the terms “include”, “have”, “consist of”, and the like are used in the present specification, other parts may be added unless “only” is used. When a component is expressed in the singular, it includes a case where the plural is included unless there is a specifically explicit description.

When interpreting components, it is interpreted as including the error range even if there is no separate explicit description.

The individual features of the various embodiments of the present disclosure may be partially or wholly coupled or combined with each other, and as can be fully understood by those skilled in the art, various technical connections and operations are possible, and each embodiment may be implemented independently of each other or may be implemented together in a related relationship.

For clarity in the interpretation of the present specification, the terms used in the present specification are defined below.

The term “subject” as used herein may mean any subject seeking to receive information on the diagnosis of aortic stenosis, or further, severity thereof, from a cardiac ultrasound image. Meanwhile, the object disclosed in the present specification may be any mammal other than a human, but is not limited thereto.

As used herein, the term “cardiac ultrasound image” refers to a cardiac ultrasound image of a subject, which may be a still cut image of a single frame or a moving image composed of multiple frames.

In this case, the cardiac ultrasound image may be a 2D or 3D image.

According to an aspect of the present disclosure, the cardiac ultrasound image may be a B-mode cardiac ultrasound image. More specifically, the cardiac ultrasound image may be a B-mode PLAX (parasternal long-axis) cross-sectional image or a B-mode PSAX (parasternal short-axis) AV (aortic valve) level cross-sectional image, which can provide information on the aortic stenosis.

However, the cardiac ultrasound images are not limited thereto and may include Doppler cardiac ultrasound images, color Doppler cardiac ultrasound images, or M-mode cardiac ultrasound images that enable determination of measurements for diagnosing aortic stenosis.

As used herein, the term “anatomical structure” may refer to an anatomical structure of the heart that can be identified in the cardiac ultrasound image.

In at least one implementation, the anatomical structure may include one region selected from a right ventricle anterior wall, a right ventricle (RV), an anterior wall of aorta, an aorta, a posterior wall of aorta, a left atrium (LA), and a posterior wall of LA. However, the anatomical structure is not limited thereto.

As used herein, the terms “measurement” or “parameter” may refer to a measurable value of thickness, diameter, or the like for a cardiac structure, and may be used interchangeably with each other in the present specification. In particular, as used herein, the measurement may be a parameter associated with the diagnosis of aortic stenosis, and may reflect the features of a cascade of progression in the severity of the stenosis.

In at least one implementation, the measurement may be at least one of Vmax (peak aortic jet velocity), mPG (mean pressure gradient), AVA (aortic valve area), EOA (effective orifice area), DI (dimensionless index), and indexed AVA. Preferably, the measurement may be the Vmax, mPG and AVA, but is not limited thereto.

Meanwhile, the Vmax refers to the maximum velocity of blood flow through an aortic valve, and the mPG refers to the mean pressure difference of blood flow through the aortic valve. Furthermore, the AVA refers to the area of the aortic valve, and the EOA refers to the effective opening area. DI refers to the ratio of the velocity through the left ventricular outflow tract (LVOT) and the aortic valve, and the indexed AVA refers to AVA adjusted for the body surface area (BSA).

The term “severity of aortic stenosis” as used herein may mean the degree of progression of aortic stenosis, and may include normal, AV sclerosis, mild, moderate, and severe.

That is, the prediction and classification of the severity of aortic stenosis may be interpreted as meaning “diagnosis of aortic stenosis or normal”.

According to various embodiments of the present disclosure, a prediction model trained to classify the severity of aortic stenosis based on the cardiac ultrasound image is presented.

As used herein, the term “prediction model” may be a model configured to take as input a cardiac ultrasound image and output the severity of aortic stenosis.

More specifically, the prediction model may be a model trained to extract features from the cardiac ultrasound image and predict and output the severity of aortic stenosis from the features.

In various embodiments, the prediction model may be configured to output a score corresponding to the severity of aortic stenosis.

That is, the medical staff may understand the severity of a subject in more detail by providing the severity score through the prediction model. For example, for subjects classified with the same severity, it is possible to distinguish between a severity close to severe and a severity close to mild.

However, it is not limited thereto, and the prediction model may be a model trained to classify the severity into five classes such as normal, AV sclerosis, mild, moderate, and severe. More specifically, the prediction model includes an output layer consisting of a plurality of nodes, where the number of the plurality of nodes may correspond to a predetermined number of severities (the number of classes).

Furthermore, the prediction model may output the severity graphically, with the severity being color-coded according to degree.

In at least one implementation, the prediction model may be a model trained to input the cardiac ultrasound image, extract the features, segment the anatomical structure of the heart based on the extracted features, and classify the severity therefrom.

That is, the prediction model may include a feature extraction module that extracts features from the cardiac ultrasound image and a segmentation module that segments the anatomical structure based on the features. In this case, the feature extraction module may correspond to an encoder, and the segmentation module may correspond to a decoder, but they are not limited thereto.

In at least one implementation, the prediction model may be a model trained to input the cardiac ultrasound image, extract features, segment the anatomical structures of the heart based on the extracted features, determine the cardiac measurement based on the extracted features, and classify the severity of aortic stenosis based on the determined cardiac measurement and the segmented results.

In this case, the prediction model may include a network module trained to determine a value of at least one cardiac measurement among the Vmax, mPG, AVA, EOA, DI, and indexed AVA based on features extracted from the cardiac ultrasound image.

For example, the prediction model may include a network module configured to take features extracted from the cardiac ultrasound image as input and output a Vmax value, a network module configured to take the extracted features as input and output an mPG value, and a network module configured to take the extracted features as input and output an AVA value.

In various embodiments, the prediction model may include a fusion layer. That is, measurements determined from each of a plurality network modules are fused in the fusion layer and finally passed through the output layer to classify the severity of the aortic stenosis.

In more diverse embodiments, measurements and heart segmentation results determined from each of the plurality of network modules may be fused through the fusion layer, and finally the severity of aortic stenosis may be classified through an output layer.

Meanwhile, the prediction model may be based on at least one algorithm selected from among DenseNet-121, U-net, VGG net, DenseNet, and fully convolutional network (FCN) with encoder-decoder structure, deep neural network (DNN) such as SegNet, DeconvNet, and DeepLAB V3+, transformer such as Lawin+, SegFormer, and Swin, SqueezeNet, Alexnet, ResNet18, MobileNet-v2, GoogLeNet, Resnet-v2, Resnet50, RetinaNet, Resnet101, Inception-v3, HRNet, ResNeXt, EfficientNet. Furthermore, the prediction model may be an ensemble model based on at least two algorithm models of the above-mentioned algorithms. However, the present disclosure is not limited thereto.

The term “classification model” as used in the present specification may mean an artificial intelligence-based model that is trained to automatically classify whether the cardiac ultrasound image corresponds to bicuspid aortic stenosis or tricuspid aortic stenosis by receiving the cardiac ultrasound image as input. For example, the classification model may be a deep learning-based classification neural network that has trained visual features such as the morphological structure, opening and closing pattern, or the like of a valve in a 2D or 3D cardiac ultrasound image.

However, the present disclosure is not limited thereto, and the classification model may be based on a recurrent neural network or a time series model capable of processing temporal features including the movement of the valve.

The term “first prediction model” used in the present specification is a prediction model applied when the classified pathological condition is the bicuspid aortic stenosis, and may mean an artificial neural network-based model pre-trained to predict the severity of the bicuspid aortic stenosis from the cardiac ultrasound images.

For example, the first prediction model may be configured to estimate severity by reflecting morphological and further functional characteristics unique to the bicuspid valve, such as valve opening area and blood flow velocity, but is not limited thereto.

The term “second prediction model” used in the present specification refers to a prediction model applied when the classified pathological condition is the tricuspid aortic stenosis, and may mean an artificial neural network-based model designed to predict the severity of the pathological condition by training in a form specialized for cardiac ultrasound images of the tricuspid valve structure.

For example, the second prediction model may be a model trained by reflecting features such as symmetrical closure pattern and uniform valve movement according to the tricuspid valve structure, but is not limited thereto.

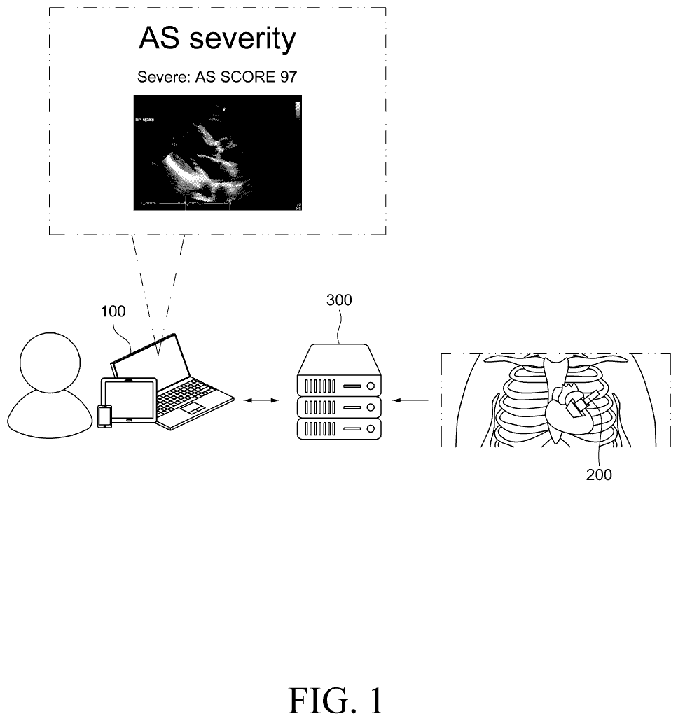

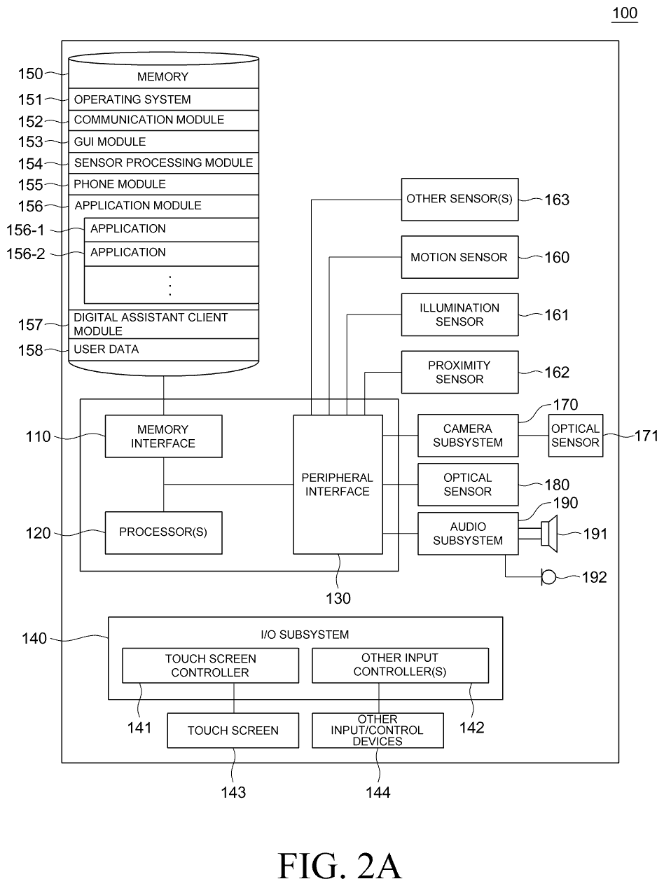

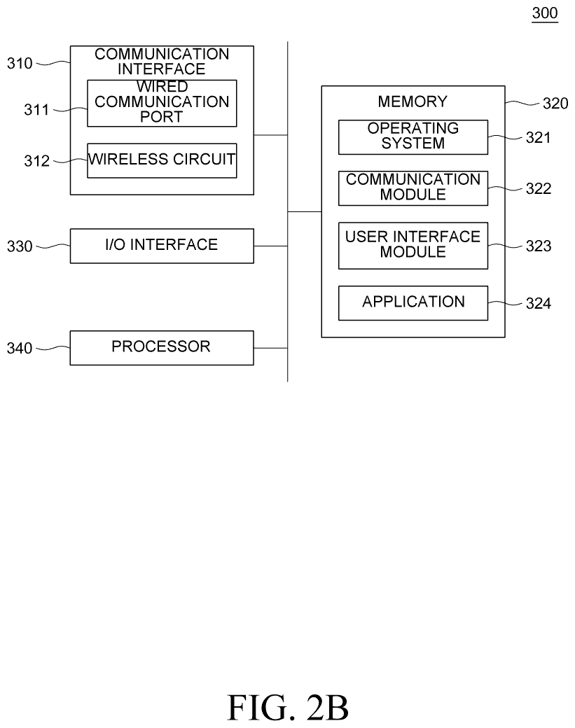

Hereinafter, with reference to FIGS. 1 and 2A and 2B, an information providing system for the severity of aortic stenosis using a device for providing information on the severity of aortic stenosis according to one embodiment of the present disclosure and a device for providing information on the severity of aortic stenosis will be described.

FIG. 1 illustrates a system for providing information on the severity of aortic stenosis using a device for providing information on the severity of aortic stenosis according to one embodiment of the present disclosure. FIG. 2A illustrates an exemplary configuration of a medical staff device for receiving information on the severity of aortic stenosis according to one embodiment of the present disclosure. FIG. 2B illustrates an exemplary configuration of a server for providing information on the severity of aortic stenosis according to one embodiment of the present disclosure.

First, referring to FIG. 1, an information providing system 1000 may be a system configured to provide information related to the severity of aortic stenosis based on a cardiac ultrasound image of a subject. In this case, the information providing system 1000 may be configured with a medical staff device 100 that receives information related to the severity of aortic stenosis, an ultrasound imaging diagnosis device 200 that provides the cardiac ultrasound image, and an information providing server 300 that generates the information related to the severity of aortic stenosis based on the received cardiac ultrasound image.

In various embodiments of the present disclosure, information providing server 300 may be mounted on the ultrasonic imaging diagnosis device 200, in which case, various information related to measurements may be displayed on a display unit (not illustrated) of the ultrasonic imaging diagnosis device 200.

That is, a user may use ultrasound imaging diagnosis device 200 to check information related to the severity of the aortic stenosis simultaneously with the diagnosis.

In various embodiments, medical staff device 100 is an electronic device that provides a user interface for presenting the information related to the severity of the aortic stenosis, and may include at least one of a smart phone, a tablet personal computer (PC), a laptop, and/or a PC.

Medical staff device 100 may receive guidelines for obtaining B-mode images to determine the severity of the aortic stenosis for the subject from an information providing server 300 and display the received results through a display unit (not illustrated).

Information providing server 300 may include a general-purpose computer, laptop, and/or data server that performs various operations to determine the information related to the severity of the aortic stenosis based on the cardiac ultrasound image provided from ultrasound imaging diagnosis device 200, such as an ultrasound diagnosis device. In this case, information providing server 300 may be a device for accessing a web server providing a web page or a mobile web server providing a mobile website but is not limited thereto.

More specifically, information providing server 300 receives the cardiac ultrasound image from ultrasound imaging diagnosis device 200, extracts the features from the received cardiac ultrasound image, and performs a prediction for classifying the severity from the features. In this case, information providing server 300 may extract the features from the cardiac ultrasound image using the prediction model and perform a prediction for classifying the severity of the aortic stenosis.

Furthermore, information providing server 300 may determine the severity of the aortic stenosis based on the anatomical structure.

Information providing server 300 may provide the severity (for example, severity score) of the aortic stenosis to medical staff device 100.

The information provided from information providing server 300 in this way may be provided as a web page through a web browser installed on a medical staff device 100 or may be provided in the form of an application or program. In various embodiments, such data may be provided in a form included in a platform in a client-server environment.

Next, the components of information providing server 300 of the present disclosure will be specifically described with reference to FIGS. 2A and 2B.

First, referring to FIG. 2A, medical staff device 100 may include a memory interface 110, one or more processors 120, and a peripheral interface 130. Various components within medical staff device 100 may be connected by one or more communication buses or signal lines.

Memory interface 110 may be connected to a memory 150 to transmit various data to processor 120. Here, memory 150 may include at least one type of storage medium among flash memory type, hard disk type, multimedia card micro type, card type memory (for example, SD or XD memory, or the like), RAM, SRAM, ROM, EEPROM, PROM, network storage, cloud, and blockchain data.

In various embodiments, memory 150 may store at least one of an operating system 151, a communication module 152, a graphical user interface (GUI) module 153, a sensor processing module 154, a phone module 155, and an application module 156. Specifically, operating system 151 may include instructions for processing basic system services and instructions for performing hardware operations. Communication module 152 may communicate with at least one of other devices, computers, and servers. Graphical user interface (GUI) module 153 may process a graphical user interface. Sensor processing module 154 may process sensor-related functions (for example, processing voice input received using one or more microphones 192). Phone module 155 may process phone-related functions. Application module 156 may perform various functions of a user application, such as electronic messaging, web browsing, media processing, navigation, imaging, and other processing functions. In addition, medical staff device 100 may store one or more software applications 156-1 and 156-2 (for example, information providing applications) associated with a type of service in memory 150.

In various embodiments, memory 150 may store a digital assistant client module 157 (hereinafter, DA client module), and accordingly, may store instructions for performing functions of a client side of the digital assistant and various user data 158.

Meanwhile, DA client module 157 may obtain the user's voice input, text input, touch input, and/or gesture input through various user interfaces (for example, I/O subsystem 140) provided in medical staff device 100.

In addition, DA client module 157 may output data in an audiovisual and tactile form. For example, DA client module 157 may output data composed of a combination of at least two or more of voice, sound, notification, text message, menu, graphic, video, animation, and vibration. Moreover, DA client module 157 may communicate with a digital assistant server (not illustrated) using a communication subsystem 180.

In various embodiments, DA client module 157 may collect additional information about the surroundings of medical staff device 100 from various sensors, subsystems, and peripheral devices to construct a context associated with the user input. For example, DA client module 157 may provide context information along with the user input to a digital assistant server to infer the user's intent. Here, the context information that may accompany the user input may include sensor information, such as lighting, ambient noise, ambient temperature, images of the surrounding environment, video, or the like. In another example, the context information may include the physical state of medical staff device 100 (for example, device orientation, device position, device temperature, power level, speed, acceleration, motion pattern, cellular signal strength, or the like). For another example, the context information may include information related to the software state of medical staff device 100 (for example, processes running on medical staff device 100, installed programs, past and current network activity, background services, error logs, resource usage, or the like).

In various embodiments, memory 150 may include additional or deleted instructions, and may further include additional configurations other than the configurations illustrated in FIG. 2A of medical staff device 100 or may exclude some configurations.

Processor 120 may control the overall operation of medical staff device 100 and may execute various commands to implement an interface that provides information related to the severity of aortic stenosis by running an application or program stored in memory 150.

Processor 120 may correspond to a computing device such as a central processing unit (CPU) or an application processor (AP). In addition, processor 120 may be implemented in the form of an integrated chip (IC) such as a system on chip (SoC) in which various computing devices such as a neural processing unit (NPU) are integrated.

Peripheral interface 130 may be connected to various sensors, subsystems, and peripheral devices, and may provide data so that medical staff device 100 can perform various functions. Here, it can be understood that the function performed by medical staff device 100 is performed by processor 120.

Peripheral interface 130 may receive data from a motion sensor 160, a light sensor (illumination sensor) 161, and a proximity sensor 162, through which medical staff device 100 may perform orientation, light, and proximity detection functions, or the like. For another example, peripheral interface 130 may receive data from other sensors 163 (e.g., positioning system-GPS receiver, temperature sensor, biometric sensor), through which medical staff device 100 may perform functions related to other sensors 163.

In various embodiments, medical staff device 100 may include a camera subsystem 170 connected to peripheral interface 130 and an optical sensor 171 connected thereto, which enables medical staff device 100 to perform various photographing functions, such as taking photographs and recording video clips.

In various embodiments, medical staff device 100 may include communication subsystem 180 coupled with a peripheral interface 130. Communication subsystem 180 may be comprised of one or more wired/wireless networks and may include various communication ports, radio frequency transceivers, and optical transceivers.

In various embodiments, medical staff device 100 includes an audio subsystem 190 coupled to peripheral interface 130, the audio subsystem 190 including one or more speakers 191 and one or more microphones 192, such that medical staff device 100 may perform voice-activated functions, such as speech recognition, voice duplication, digital recording, and telephony.

In various embodiments, medical staff device 100 may include an I/O subsystem 140 coupled to peripheral interface 130. For example, I/O subsystem 140 may control a touch screen 143 included in medical staff device 100 via a touch screen controller 141. As an example, touch screen controller 141 may detect a user's contact and movement or cessation of contact and movement using any one of a plurality of touch sensing technologies, such as capacitive, resistive, infrared, surface acoustic wave technology, proximity sensor array, and the like. As another example, I/O subsystem 140 may control other input/control devices 144 included in medical staff device 100 via other input controller(s) 142. As an example, other input controller(s) 142 may control one or more buttons, rocker switches, thumb-wheels, infrared ports, USB ports, and pointer devices such as a stylus.

Next, referring to FIG. 2B, information providing server 300 may include a communication interface 310, a memory 320, an I/O interface 330, and a processor 340, each component of which may communicate with each other through one or more communication buses or signal lines.

Communication interface 310 may be connected to medical staff device 100 and ultrasound imaging diagnosis device 200 via a wired/wireless communication network to exchange data. For example, communication interface 310 may receive the cardiac ultrasound image from ultrasound imaging diagnosis device 200, determine the severity of aortic stenosis or a guideline for obtaining the same from the received cardiac ultrasound image, and transmit the determined guideline to medical staff device 100.

Meanwhile, communication interface 310 that enables transmission and reception of such data includes a communication port 311 and a wireless circuit 312, in which wired communication port 311 may include one or more wired interfaces, for example, Ethernet, universal serial bus (USB), FireWire, or the like. In addition, wireless circuit 312 may transmit and receive data with an external device via an RF signal or an optical signal. In addition, the wireless communication may use at least one of a plurality of communication standards, protocols, and technologies, such as GSM, EDGE, CDMA, TDMA, Bluetooth, Wi-Fi, VoIP, Wi-MAX, or any other suitable communication protocol.

Memory 320 may store various data used in information providing server 300. For example, memory 320 may store the cardiac ultrasound image or the prediction model trained to classify the severity of the aortic stenosis from the cardiac ultrasound image.

In various embodiments, memory 320 may include a volatile or nonvolatile storage medium capable of storing various data, commands, and information. For example, memory 320 may include at least one type of storage medium among flash memory type, hard disk type, multimedia card micro type, card type memory (such as SD or XD memory), RAM, SRAM, ROM, EEPROM, PROM, network storage, cloud, and blockchain data.

In various embodiments, memory 320 may store at least one configuration among an operating system 321, a communication module 322, a user interface module 323, and one or more applications 324.

Operating system 321 (for example, an embedded operating system such as LINUX, UNIX, MAC OS, WINDOWS, VxWorks, or the like) may include various software components and drivers for controlling and managing general system operations (for example, memory management, storage device control, power management, or the like) and may support communication between various hardware, firmware, and software components.

Communication module 322 may support communication with other devices through communication interface 310. The communication module 322 may include various software components for processing data received by wired communication port 311 or wireless circuit 312 of communication interface 310.

User interface module 323 may receive user's requests or inputs from a keyboard, touch screen, microphone, or the like through an I/O interface 330, and provide a user interface on the display.

Application 324 may include a program or module configured to be executed by one or more processors 340. Here, the application for providing information related to the severity of aortic stenosis may be implemented on a server farm.

I/O interface 330 may connect an input/output device (not illustrated) of information providing server 300, for example, at least one of a display, a keyboard, a touch screen, and a microphone, to user interface module 323. I/O interface 330 may receive user input (for example, voice input, keyboard input, touch input, or the like) together with user interface module 323 and process a command according to the received input.

Processor 340 may be connected to communication interface 310, memory 320, and I/O interface 330 to control the overall operation of information providing server 300, and may perform various commands for providing information through an application or program stored in memory 320.

Processor 340 may correspond to a computing device such as a central processing unit (CPU) or an application processor (AP). In addition, processor 340 may be implemented in the form of an integrated chip (IC) such as a system on chip (SoC) in which various computing devices are integrated. Alternatively, processor 340 may include a module for calculating an artificial neural network model such as a neural processing unit (NPU).

In various embodiments, processor 340 may be configured to extract the features within the cardiac ultrasound images using the prediction model, and optionally automatically determine the anatomical structures and cardiac measurements to provide information on the aortic stenosis. Optionally, processor 340 may be configured to provide a guideline for image acquisition that enable severity classification of the aortic stenosis from the cardiac ultrasound images.

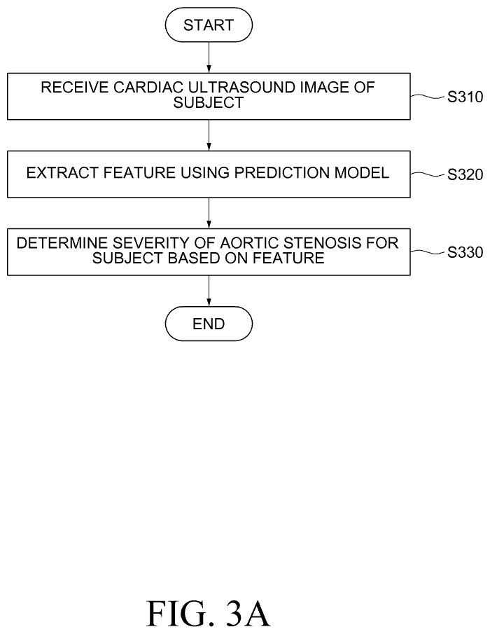

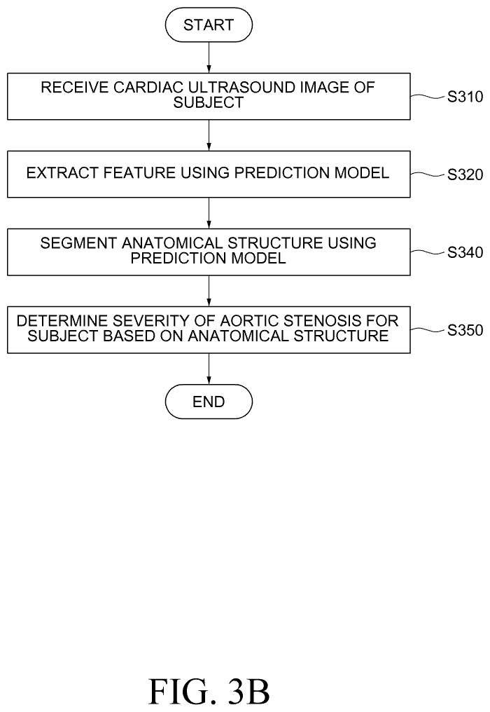

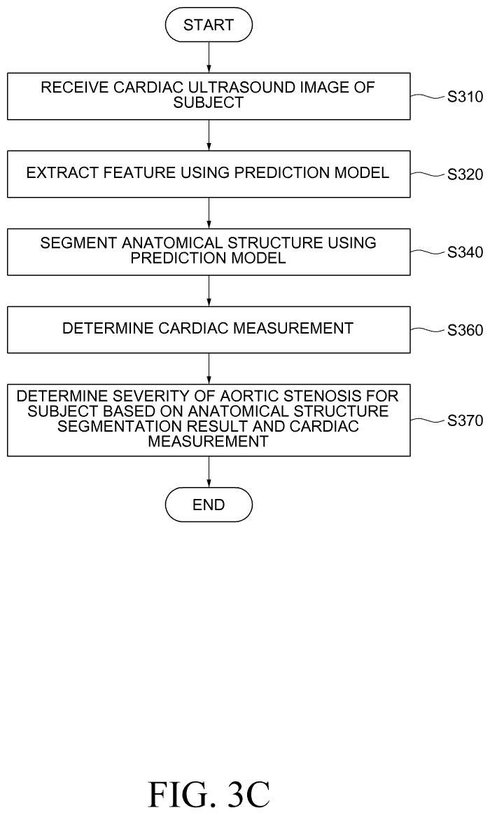

Hereinafter, a method for providing information on the severity of aortic stenosis according to one embodiment of the present disclosure will be specifically described with reference to FIGS. 3A to 3C, FIGS. 4A and 4B, and FIG. 5.

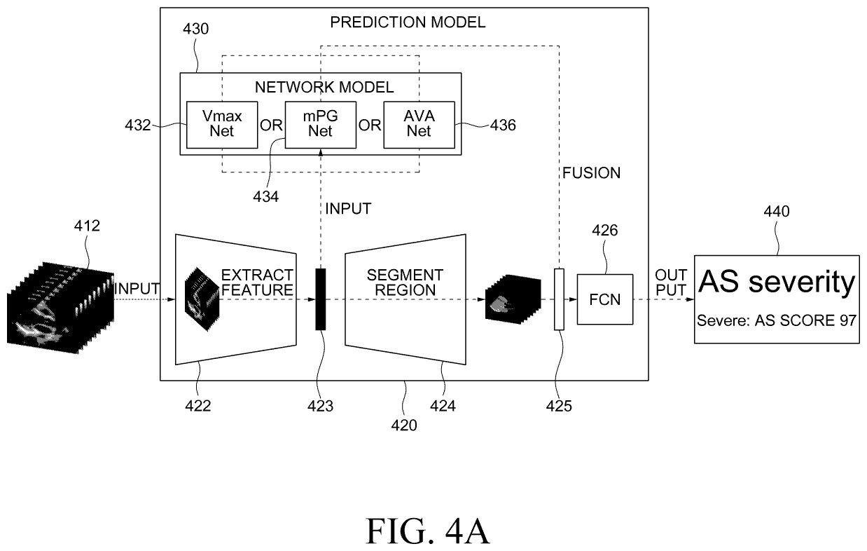

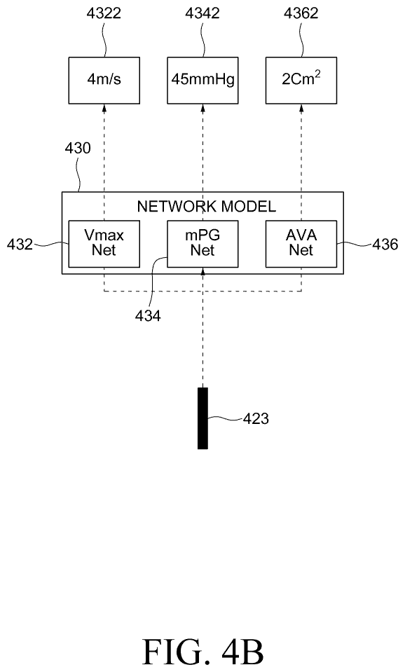

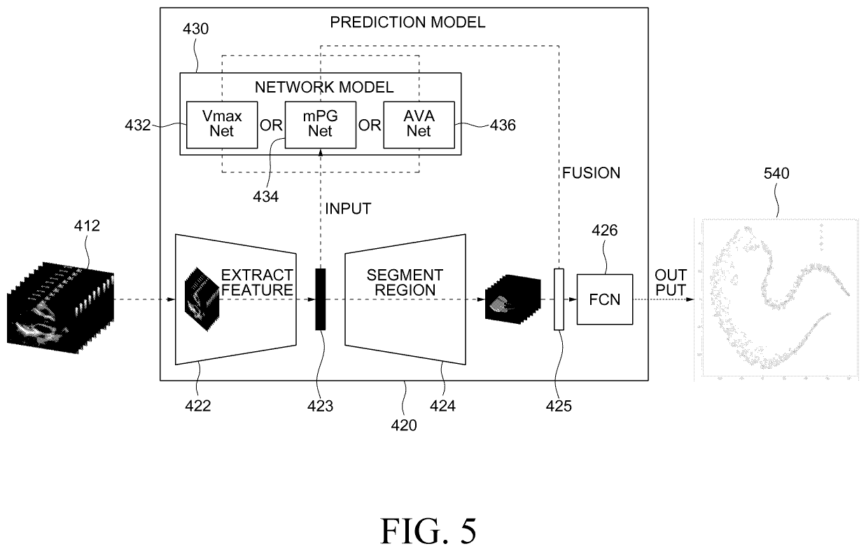

FIGS. 3A to 3C illustrate a procedure of the method for providing information on the severity of the aortic stenosis according to one embodiment of the present disclosure. FIGS. 4A and 4B and FIG. 5 illustrate an example of the procedure of the method for providing information on the severity of the aortic stenosis according to one embodiment of the present disclosure.

First, referring to FIG. 3A, the information providing procedure according to one embodiment of the present disclosure is as follows. First, the cardiac ultrasound image of the subject is received (S310). Then, the features are determined from the cardiac ultrasound image by prediction model (S320). Then, measurable parameters are determined based on extracted features (S330).

More specifically, in step (S310) where the cardiac ultrasound image is received, a cardiac ultrasound image of a target area, that is, a heart area, may be received.

According to an aspect of the present disclosure, in step (S310) of receiving the cardiac ultrasound image, the cardiac ultrasound image of a still cut in B-mode or the cardiac ultrasound moving image including a plurality of frames may be received.

According to an aspect of the present disclosure, in step (S310) of receiving the cardiac ultrasound image, a B-mode PLAX (parasternal long-axis) cross-sectional view or a B-mode PSAX AV level cross-sectional view may be received.

However, the present disclosure is not limited thereto, and a wider variety of cardiac ultrasound images capable of determining measurements may be received at step (S310) of receiving the cardiac ultrasound image.

In various embodiments, after step (S310) of receiving the cardiac ultrasound image, a step of evaluating the quality of the cardiac ultrasound image and, depending on the quality of the evaluated cardiac ultrasound image, a step of providing the guideline to acquire the B-mode PLAX cross-sectional view or the B-mode PSAX AV level cross-sectional view may be further performed.

Next, the step (S320) is performed in which the features are extracted from the cardiac ultrasound image.

According to an aspect of the present disclosure, step (S320) of extracting the features may be performed by the prediction model trained to extract the features from the cardiac ultrasound image by inputting the received cardiac ultrasound image and classify the severity of the aortic stenosis.

Next, the severity of the aortic stenosis for the subject is determined based on the features extracted by prediction model (S330).

Meanwhile, in various embodiments, the severity of the aortic stenosis may be determined based on the anatomical features of the heart.

For example, referring to FIG. 3B, after step (S320) of extracting features, the anatomical structures of the heart are segmented based on the extracted features using prediction model (S340), and the severity of aortic stenosis for the subject may be determined based on the segmented anatomical structures using prediction model (S350).

That is, the prediction model includes a feature extraction module that extracts features from the cardiac ultrasound image and a segmentation module that segments the anatomical structures based on the features. Through the segmentation module, the anatomical structures of the heart are segmented, and the severity of the aortic stenosis for the subject may be classified based on the segmentation result.

In this case, the prediction model may segment at least one region selected from the right ventricle anterior wall, right ventricle (RV), anterior wall of aorta, aorta, posterior wall of aorta, left atrium (LA), and posterior wall of LA in the cardiac ultrasound image.

In various embodiments of the present disclosure, the severity of the aortic stenosis may be determined based on parameters obtainable from the cardiac ultrasound images. In this case, the cardiac parameters, that is, measurements, may be data reflecting the characteristic of the stenosis severity progressing serially.

For example, referring to FIG. 3B, after step (S320) of extracting the features, the anatomical structures of the heart are segmented based on the extracted features using prediction model (S340), the cardiac measurements are determined based on extracted features (S360), and the severity of the aortic stenosis for the subject may be classified and determined based on the anatomical structures segmented by the prediction model and measurements (S370).

In this case, the cardiac measurement may be at least one of Vmax (peak aortic jet velocity), mPG (mean pressure gradient), AVA (aortic valve area), EOA (effective orifice area), DI (dimensionless index) and indexed AVA, and the prediction model may include at least one network module trained to determine at least one cardiac measurement by inputting the features.

In various embodiments, the plurality of the network modules may be provided, and the prediction model further includes a fusion layer and an output layer, such that cardiac measurements determined from each of the plurality of network modules are fused through the fusion layer, and the severity of the aortic stenosis may be output and classified based on the fused cardiac measurement results through the output layer.

In a specific example, referring to FIG. 4A together, in step (S310) of receiving the cardiac ultrasound image, a received cardiac ultrasound image 412 is input to a prediction model 420. In this case, a feature 423 for cardiac ultrasound image 412 is extracted through a feature extraction module 422, and anatomical structures in cardiac ultrasound image 412 are determined based on feature 423 through a segmentation module 424. In this case, the feature extraction module may correspond to an encoder, and the segmentation module may correspond to a decoder, but they are not limited thereto.

Optionally, extracted feature 423 is input to a network module 430 built to predict each cardiac parameter. Meanwhile, network module 430 may include various combinations of network modules trained to determine the value of at least one cardiac measurement among Vmax, mPG, AVA, EOA, DI, and indexed AVA based on the features extracted from the cardiac ultrasound image.

More specifically, referring to FIG. 4B together, the network module 430 may be composed of a network module 432 configured to input features extracted from the cardiac ultrasound image and output a Vmax value, a network module 434 configured to input extracted features 423 and output an mPG value, and a network module 436 configured to input the extracted features and output an AVA value.

In this case, network modules 432, 434, and 436 may output values 4322, 4342, and 4362 corresponding to each parameter.

Returning to FIG. 4A again, through a fusion layer 425, measurements determined from each of the plurality of network modules and further the heart segmentation results are fused, and finally, through the output layer, the severity of aortic stenosis may be classified.

In more diverse embodiments, at step (S370) in which the severity of the aortic stenosis is determined, the severity score corresponding to the severity of the aortic stenosis may be output by the prediction model.

That is, through the output layer (not illustrated), a severity score 440 corresponding to the severity of the aortic stenosis may be output and provided. By providing the severity score, it is possible to provide information on more detailed severity for the subject. For example, for subjects classified with the same severity, it is possible to distinguish between a severity close to severe and a severity close to mild.

However, the output format of the prediction model is not limited to this.

For example, the prediction model may be configured to classify the severity into five classes such as normal, AV sclerosis, mild, moderate, and severe.

Furthermore, referring to FIG. 5 further, the prediction model may output graphic data 540 that visualizes the severity so that the severity is distinguished by color according to the degree. In this case, graphic data 540 may be displayed with colors that gradually change according to the severity, as parameters that reflect the characteristic of the severity of stenosis progressing serially are reflected in the prediction.

That is, the severity of aortic stenosis may be confirmed using only the cardiac ultrasound images by using the prediction model, and thus, the information on the severity of the aortic stenosis may be provided without going through the process of comparing the degree of stenosis based on the cardiac ultrasound measurements with the guideline criteria.

Thus, when there are limitations in the work to obtain measurements, information on the severity of aortic stenosis may be provided, enabling quick and accurate decision making.

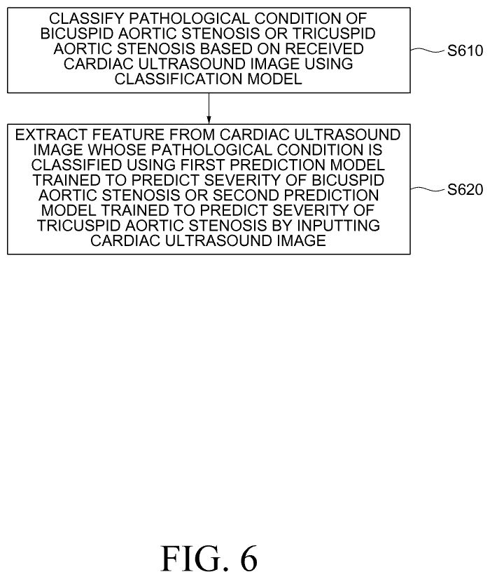

Meanwhile, according to more various embodiments of the present disclosure, as illustrated in FIG. 6, a step (S610) is performed in which, based on the cardiac ultrasound image received from the subject, the classification is performed to determine whether the image corresponds to bicuspid aortic stenosis or tricuspid aortic stenosis. In this case, a pre-trained classification model may be used to classify a pathological condition by inputting the cardiac ultrasound image received in classification step (S610).

That is, based on the anatomical structure, movement pattern, and visual features of the aortic valve observed in the cardiac ultrasound image by the classification model, it is automatically classified whether the cardiac ultrasound image corresponds to the pathological condition of the bicuspid aortic stenosis or the pathological condition of the tricuspid aortic stenosis.

Next, in a step (S620) of extracting the features, different prediction models are selectively applied depending on the classified pathological condition. More specifically, when the pathological condition is classified as the bicuspid aortic stenosis, a first prediction model trained to predict the severity of the bicuspid aortic stenosis is applied, and when the pathological condition is classified as the tricuspid aortic stenosis, a second prediction model trained to predict the severity of the tricuspid aortic stenosis is applied. The plurality of prediction models are models trained by considering the anatomical and functional features of the cardiac ultrasound images specialized for each pathological condition, and may be configured to automatically extract prediction features suitable for the corresponding pathological condition from the input image.

That is, the information providing method according to various embodiments of the present disclosure provides customized predictions that take into account the differences in pathological conditions such as bicuspid and tricuspid valves, thereby allowing for more accurate and precise prediction of the severity of the aortic stenosis by reflecting anatomical diversity according to the pathological conditions.

This may contribute to reducing prediction errors that may occur in single-model-based predictions and increasing the reliability of clinical decision-making.

### Evaluation: Evaluation of Prediction Model

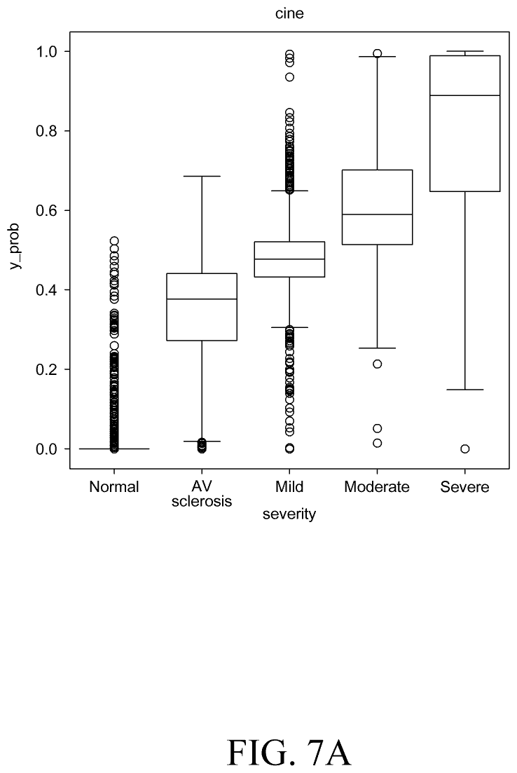

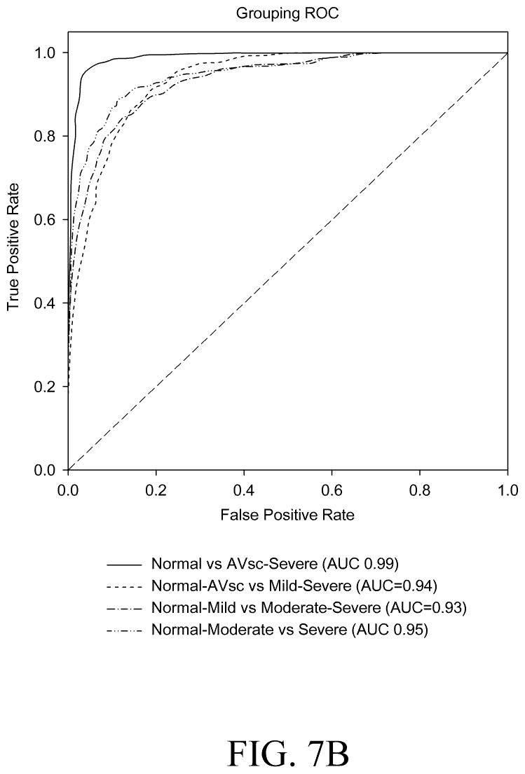

Hereinafter, with reference to FIGS. 7A and 7B, the evaluation results of the prediction model applied to various embodiments of the present disclosure will be described. FIGS. 7A and 7B illustrate the evaluation results of the prediction model applied to the information providing method according to various embodiments.

In the present evaluation, the prediction model may be a model trained to extract features based on PLAX cross-sectional views of the B-mode image and B-mode PSAX AV level cross-sectional views, and classify the severity into five classes such as normal, AV sclerosis, mild, moderate, and severe, based on the segmented anatomical features and parameters.

Referring to FIG. 7A, the prediction model clearly classifies the subjects into five severity groups.

Referring to FIG. 7B together, the prediction model is illustrated to have a high AUC of 0.99 in classifying the normal group and the AV sclerosis to severe arterial stenosis groups, and an AUC of 0.94 in classifying the normal-AV sclerosis group and the mild to severe groups. Furthermore, the prediction model is illustrated to have an AUC of 0.93 in classifying the normal to mild groups and the moderate to severe groups, and an AUC of 0.95 in classifying the normal to moderate groups and the severe group.

These results may imply that the prediction model has excellent diagnostic performance, as it can predict not only the diagnosis of aortic stenosis but also the severity of the aortic stenosis with high accuracy.

That is, the present disclosure may provide classification results for the presence or absence of aortic stenosis and the severity thereof using only B-mode images, regardless of the skill level of the medical staff, and may contribute to faster and more accurate decision-making and treatment plan establishment at the image analysis stage.

Although the embodiments of the present disclosure have been described in more detail with reference to the attached drawings, the present disclosure is not necessarily limited to these embodiments, and various modifications may be made without departing from the technical idea of the present disclosure. Accordingly, the embodiments disclosed in the present disclosure are not intended to limit the technical idea of the present disclosure, but to explain it, and the scope of the technical idea of the present disclosure is not limited by these embodiments. Therefore, it should be understood that the embodiments described above are exemplary in all aspects and not restrictive. The protection scope of the present disclosure should be interpreted by the following claims, and all technical ideas within a scope equivalent thereto should be interpreted as being included in the scope of the rights of the present disclosure.

What is claimed is:

1. A method for providing information on severity of aortic stenosis implemented by a processor, the method comprising:
receiving a cardiac ultrasound image of a subject;
extracting features from the received cardiac ultrasound image using a prediction model trained to predict the severity of the aortic stenosis by inputting the cardiac ultrasound image; and
determining the severity of the aortic stenosis for the subject based on the extracted features using the prediction model.

2. The method according to claim 1, further comprising:
after the extracting the features,
segmenting an anatomical structure of a heart based on the extracted features using the prediction model; and
determining the severity of the aortic stenosis for the subject based on the segmented anatomical structure using the prediction model.

3. The method according to claim 1, further comprising:
after the extracting the features,
segmenting an anatomical structure of a heart based on the extracted features using the prediction model;
determining a cardiac measurement based on the extracted features; and
determining the severity of the aortic stenosis for the subject based on the segmented anatomical structure and the cardiac measurement using the prediction model.

4. The method according to claim 3, wherein a plurality of the cardiac measurements is provided, and wherein the prediction model includes a plurality of network modules trained to determine a value corresponding to each of the plurality of cardiac measurements by inputting the features.

5. The method according to claim 3, wherein the cardiac measurement is at least one of Vmax (peak aortic jet velocity), mPG (mean pressure gradient), AVA (aortic valve area), EOA (effective orifice area), DI (dimensionless index), or indexed AVA.

6. The method according to claim 1, wherein the cardiac ultrasound image is a B-mode PLAX (parasternal long-axis) cross-sectional image or a B-mode PSAX (parasternal short-axis) AV (aortic valve) level cross-sectional image.

7. The method according to claim 6, further comprising:
after the receiving,
evaluating a quality of the cardiac ultrasound image; and
providing a guideline to acquire the B-mode PLAX cross-sectional image or the B-mode PSAX AV level cross-sectional image, depending on the evaluated quality of the cardiac ultrasound image.

8. The method according to claim 1, wherein the determining the severity of the aortic stenosis includes determining a severity score corresponding to the severity of the aortic stenosis using the prediction model.

9. The method according to claim 1, further comprising:
after the receiving, classifying a pathological condition of bicuspid aortic stenosis or tricuspid aortic stenosis based on the received cardiac ultrasound image using a classification model trained to classify the pathological condition of the bicuspid aortic stenosis or tricuspid aortic stenosis by inputting the cardiac ultrasound image,
wherein the extracting the features further includes extracting features from the cardiac ultrasound image in which the pathological condition is classified by using a first prediction model trained to predict the severity of the bicuspid aortic stenosis or a second prediction model trained to predict the severity of the tricuspid aortic stenosis, by inputting the cardiac ultrasound image.

10. A method for providing information on severity of aortic stenosis implemented by a processor, the method comprising:
receiving a cardiac ultrasound image of a subject;
extracting features from the received cardiac ultrasound image using a prediction model trained to predict the severity of the aortic stenosis by inputting the cardiac ultrasound image;
segmenting an anatomical structure of a heart based on the extracted features using the prediction model;
determining a cardiac measurement based on the extracted features using the prediction model; and
determining the severity of the aortic stenosis for the subject based on the segmented anatomical structure and the cardiac measurement using the prediction model.

11. A device for providing information on severity of aortic stenosis, the device comprising:
a communication unit configured to receive a cardiac ultrasound image of a subject; and
a processor functionally connected to the communication unit,
wherein the processor is configured to:
extract features from the received cardiac ultrasound image using a prediction model trained to predict the severity of aortic stenosis by inputting the cardiac ultrasound image, and
determine the severity of the aortic stenosis for the subject based on the extracted features.

12. The device according to claim 11, wherein the processor is further configured to:
segment an anatomical structure of a heart based on the extracted features using the prediction model, and
determine the severity of the aortic stenosis for the subject based on the segmented anatomical structure using the prediction model.

13. The device according to claim 11, wherein the processor is configured to:
segment an anatomical structure of a heart based on the extracted features using the prediction model,
determine a cardiac measurement based on the extracted features, and
determine the severity of the aortic stenosis for the subject based on the segmented anatomical structure and the cardiac measurement using the prediction model.

14. The device according to claim 13, wherein a plurality of the cardiac measurements is provided, and wherein the prediction model includes a plurality of network modules trained to determine values corresponding to each of the plurality of cardiac measurements by inputting the features.

15. The device according to claim 13, wherein the cardiac measurement is at least one of: Vmax, mPG, AVA, EOA, DI, or indexed AVA.

16. The device according to claim 11, wherein the cardiac ultrasound image is a B-mode PLAX cross-sectional image or a B-mode PSAX AV level cross-sectional image.

17. The device according to claim 16, wherein the processor is further configured to:
evaluate a quality of the cardiac ultrasound image, and
provide a guideline to acquire the B-mode PLAX cross-sectional image or the B-mode PSAX AV level cross-sectional image, depending on the evaluated quality of the cardiac ultrasound image.

18. The device according to claim 11, wherein the processor is further configured to determine a severity score corresponding to the severity of the aortic stenosis using the prediction model.

19. The device according to claim 11, wherein the processor is further configured to:
classify a pathological condition of bicuspid aortic stenosis or tricuspid aortic stenosis based on the received cardiac ultrasound image using a classification model trained to classify the pathological condition of the bicuspid aortic stenosis or tricuspid aortic stenosis by inputting the received cardiac ultrasound image, and
extract the features from the cardiac ultrasound image in which the pathological condition is classified by using a first prediction model trained to predict the severity of the bicuspid aortic stenosis or a second prediction model trained to predict the severity of the tricuspid aortic stenosis, by inputting the cardiac ultrasound image.
